(19) United States
(12) Patent Application Publication
(10) Pub. No.: US 2019/0183803 A1
(43) Pub. Date: Jun. 20, 2019

(54) FREEZE-DRIED FORMULATIONS OF ANTIBACTERIAL PROTEIN

(71) Applicant: INTRON BIOTECHNOLOGY, INC., Seongnam-si, Gyeonggi-do (KR)

(72) Inventors: Seong Jun YOON, Seoul (KR); Soo Youn JUN, Seoul (KR); Gi Mo JUNG, Seoul (KR); Sang Hyeon KANG, Seoul (KR)

(73) Assignee: INTRON BIOTECHNOLOGY, INC., Seongnam-si, Gyeonggi-do (KR)

(21) Appl. No.: 16/300,567

(22) PCT Filed: Jan. 9, 2017
(86) PCT No.: PCT/IB2017/050091

§ 371 Date: Nov. 10, 2018

Related U.S. Application Data
(60) Provisional application No. 62/277,588, filed on Jan. 12, 2016.

(51) Int. Cl. A61K 9/19; A61K 47/10; A61K 47/26; A61K 47/18; A61K 38/16; A61P 31/04

(52) U.S. Cl. CPC A61K 9/19; A61K 47/10; A61P 31/04; A61K 47/18; A61K 38/16; A61K 47/26

(57) ABSTRACT

A freeze-dried formulation includes an antibacterial protein having killing activity specific to at least one of or all following species: Staphylococcus arlettae, Staphylococcus aureus, Staphylococcus auricularis, Staphylococcus carnosus, Staphylococcus carprae, Staphylococcus chromogenes, Staphylococcus cohnii, Staphylococcus delphini, Staphylococcus epidermidis, Staphylococcus equorum, Staphylococcus gallinarum, Staphylococcus hemolyticus, Staphylococcus hominis, Staphylococcus intermedius, Staphylococcus kloosii, Staphylococcus lentus, Staphylococcus lugdunensis, Staphylococcus muscae, Staphylococcus pasteuri, Staphylococcus saprophyticus, Staphylococcus warneri, and Staphylococcus xylosus; a poloxamer; a sugar, and an amino acid.

The present application claims the benefit of U.S. Provisional Application No. 62/277,588, filed on Jan. 12, 2016, which is incorporated by reference for all purposes as if fully set forth herein.

### BACKGROUND OF THE INVENTION

### Field of the Invention

The present invention relates to freeze-dried formulations of antibacterial protein, specifically antibacterial protein specific to at least one of or all following species: Staphylococcus arlettae, Staphylococcus aureus, Staphylococcus auricularis, Staphylococcus carnosus, Staphylococcus carprae, Staphylococcus chromogenes, Staphylococcus cohnii, Staphylococcus delphini, Staphylococcus epidermidis, Staphylococcus equorum, Staphylococcus gallinarum, Staphylococcus hemolyticus, Staphylococcus hominis, Staphylococcus intermedius, Staphylococcus kloosii, Staphylococcus lentus, Staphylococcus lugdunensis, Staphylococcus muscae, Staphylococcus pasteuri, Staphylococcus saprophyticus, Staphylococcus warneri, and Staphylococcus xylosus.

### Discussion of the Related Art

A bacteriophage is any one of a number of virus-like microorganisms that infect bacteria and the term is commonly used in its shortened form, “phage.” A bacteriophage having killing activity specific to Staphylococcus aureus was isolated and deposited it at Korean Agricultural Culture Collection (KACC), National Institute of Agricultural Biotechnology (NIAB) on Jun. 14, 2006 (Accession No: KACC 97001P). Although this bacteriophage is effective for the prevention and treatment of Staphylococcus aureus infections, the use of this bacteriophage has some defects.

An antibacterial protein having killing activity against Staphylococcus aureus was derived from this bacteriophage, and the antibacterial protein can be used for the prevention and treatment of disease caused by Staphylococcus aureus. See, U.S. Pat. No. 8,232,370.

Furthermore, this antibacterial protein exhibited antibacterial activity specific to all the following species: Staphylococcus arlettae, Staphylococcus aureus, Staphylococcus auricularis, Staphylococcus carnosus, Staphylococcus carprae, Staphylococcus chromogenes, Staphylococcus cohnii, Staphylococcus delphini, Staphylococcus epidermidis, Staphylococcus equorum, Staphylococcus gallinarum, Staphylococcus hemolyticus, Staphylococcus hominis, Staphylococcus intermedius, Staphylococcus kloosii, Staphylococcus lentus, Staphylococcus lugdunensis, Staphylococcus muscae, Staphylococcus pasteuri, Staphylococcus saprophyticus, Staphylococcus warneri, and Staphylococcus xylosus.

When preparing a pharmaceutical composition comprising the antibacterial protein, the composition must be formulated in such a way that the activity of the antibacterial protein is maintained for an appropriate period of time. A loss in activity or stability of the antibacterial protein may result from chemical or physical instabilities of the protein, for example, due to denaturation, aggregation, or oxidation. The composition may thus be pharmaceutically unacceptable. The use of excipients is known to increase the stability of a bioactive protein, but the stabilizing effects of these excipients is unpredictable and highly dependent of the nature of bioactive protein and the excipients.

There remains a need for formulations containing an antibacterial protein as an active ingredient, and the formulations are stable for an appropriate period of time and suitable for injection. The formulations will be useful for administration in the treatment of disease caused by bacterial infection.

### SUMMARY OF THE INVENTION

The present invention provides a freeze-dried formulation including an antibacterial protein having killing activity specific to at least one of or all following species: Staphylococcus arlettae, Staphylococcus aureus, Staphylococcus auricularis, Staphylococcus carnosus, Staphylococcus carprae, Staphylococcus chromogenes, Staphylococcus cohnii, Staphylococcus delphini, Staphylococcus epidermidis, Staphylococcus equorum, Staphylococcus gallinarum, Staphylococcus hemolyticus, Staphylococcus hominis, Staphylococcus intermedius, Staphylococcus kloosii, Staphylococcus lentus, Staphylococcus lugdunensis, Staphylococcus muscae, Staphylococcus pasteuri, Staphylococcus saprophyticus, Staphylococcus warneri, and Staphylococcus xylosus; a poloxamer; a sugar; and an amino acid.

In an aspect, the concentration of the antibacterial protein in solution before freeze-drying is about 0.1 mg/mL to about 30 mg/mL.

In another aspect, the antibacterial protein consists of the amino acid sequence of SEQ. ID. NO: 1.

In another aspect, the antibacterial protein consists of the amino acid sequence of SEQ. ID. NO: 2.

In another aspect, the antibacterial protein is a mixture of a first antibacterial protein consisting of the amino acid sequence of SEQ. ID. NO: 1 and a second antibacterial protein consisting of the amino acid sequence of SEQ. ID. NO: 2.

In another aspect, the antibacterial protein includes 15-35 mole % of the first antibacterial protein and 65-85 mole % of the second antibacterial protein.

In another aspect, the antibacterial protein includes 25 mole % of the first antibacterial protein and 75 mole % of the second antibacterial protein.

In another aspect, the concentration of the poloxamer in solution before freeze-drying is about 0.1 g/L to about 10 g/L.

In another aspect, the poloxamer is poloxamer 188.

In another aspect, the sugar is D-sorbitol.

In another aspect, the concentration of the sugar in solution before freeze-drying is about 1 g/L to about 600 g/L.

In another aspect, the amino acid is L-histidine.

In another aspect, the concentration of the amino acid in solution before freeze-drying is about 0.1 g/L to about 10 g/L.

The present invention provides an antibacterial formulation including an antibacterial protein having killing activity specific to at least one of or all following species: Staphylococcus arlettae, Staphylococcus aureus, Staphylococcus auricularis, Staphylococcus carnosus, Staphylococcus carprae, Staphylococcus chromogenes, Staphylococcus cohnii, Staphylococcus delphini, Staphylococcus epidermidis, Staphylococcus equorum, Staphylococcus gallinarum, Staphylococcus hemolyticus, Staphylococcus hominis, Staphylococcus intermedius, Staphylococcus kloosii, Staphylococcus lentus, Staphylococcus lugdunensis, Staphylococcus muscae, Staphylococcus pasteuri, Staphylococcus saprophyticus, Staphylococcus warneri, and Staphylococcus xylosus; a poloxamer; a sugar; an amino acid, and water. The antibacterial protein consists of the amino acid sequence of SEQ. ID. NO: 1, and the concentration of the antibacterial protein is about 0.1 mg/mL to about 30 mg/mL.

The present invention provides an antibacterial formulation including an antibacterial protein having killing activity specific to at least one of or all following species: Staphylococcus arlettae, Staphylococcus aureus, Staphylococcus auricularis, Staphylococcus carnosus, Staphylococcus carprae, Staphylococcus chromogenes, Staphylococcus cohnii, Staphylococcus delphini, Staphylococcus epidermidis, Staphylococcus equorum, Staphylococcus gallinarum, Staphylococcus hemolyticus, Staphylococcus hominis, Staphylococcus intermedius, Staphylococcus kloosii, Staphylococcus lentus, Staphylococcus lugdunensis, Staphylococcus muscae, Staphylococcus pasteuri, Staphylococcus saprophyticus, Staphylococcus warneri, and Staphylococcus xylosus; a poloxamer; a sugar; an amino acid; and water. The antibacterial protein consists of the amino acid sequence of SEQ. ID. NO: 2, and the concentration of the antibacterial protein is about 0.1 mg/mL to about 30 mg/mL.

The present invention provides an antibacterial formulation including an antibacterial protein having killing activity specific to at least one of or all following species: Staphylococcus arlettae, Staphylococcus aureus, Staphylococcus auricularis, Staphylococcus carnosus, Staphylococcus carprae, Staphylococcus chromogenes, Staphylococcus cohnii, Staphylococcus delphini, Staphylococcus epidermidis, Staphylococcus equorum, Staphylococcus gallinarum, Staphylococcus hemolyticus, Staphylococcus hominis, Staphylococcus intermedius, Staphylococcus kloosii, Staphylococcus lentus, Staphylococcus lugdunensis, Staphylococcus muscae, Staphylococcus pasteuri, Staphylococcus saprophyticus, Staphylococcus warneri, and Staphylococcus xylosus; a poloxamer; a sugar; an amino acid; and water. The antibacterial protein includes a first antibacterial protein consisting of the amino acid sequence of SEQ. ID. NO: 1 and a second antibacterial protein consisting of the amino acid sequence of SEQ. ID. NO: 2, and the concentration of the antibacterial protein is about 0.1 mg/mL to about 30 mg/mL.

In an aspect, the antibacterial protein includes 15-35 mole % of the first antibacterial protein and 65-85 mole % of the second antibacterial protein.

In another aspect, the antibacterial protein includes 25 mole % of the first antibacterial protein and 75 mole % of the second antibacterial protein.

In another aspect, the poloxamer is poloxamer 188.

In another aspect, the concentration of the poloxamer is about 0.1 g/L to about 10 g/L.

In another aspect, the sugar is D-sorbitol.

In another aspect, the concentration of the sugar is about 1 g/L to about 600 g/L.

In another aspect, the amino acid is L-histidine.

In another aspect, the concentration of amino acid is about 0.1 g/L to about 10 g/L.

The present application provides a method for manufacturing a freeze-dried formulation including forming a mixture consisting of an antibacterial protein having killing activity specific to at least one of or all following species: Staphylococcus arlettae, Staphylococcus aureus, Staphylococcus auricularis, Staphylococcus carnosus, Staphylococcus carprae, Staphylococcus chromogenes, Staphylococcus cohnii, Staphylococcus delphini, Staphylococcus epidermidis, Staphylococcus equorum, Staphylococcus gallinarum, Staphylococcus hemolyticus, Staphylococcus hominis, Staphylococcus intermedius, Staphylococcus kloosii, Staphylococcus lentus, Staphylococcus lugdunensis, Staphylococcus muscae, Staphylococcus pasteuri, Staphylococcus saprophyticus, Staphylococcus warneri, and Staphylococcus xylosus; a poloxamer; a sugar; and an amino acid, and subjecting the mixture to lyophilization.

In an aspect, the concentration of the antibacterial protein in the mixture before lyophilization is about 0.1 mg/mL to about 30 mg/mL.

In another aspect, the antibacterial protein consists of the amino acid sequence of SEQ. ID. NO: 1.

In another aspect, the antibacterial protein consists of the amino acid sequence of SEQ. ID. NO: 2.

In another aspect, the antibacterial protein is a mixture of a first antibacterial protein consisting of the amino acid sequence of SEQ. ID. NO: 1 and a second antibacterial protein consisting of the amino acid sequence of SEQ. ID. NO: 2.

In another aspect, the antibacterial protein includes 15-35 mole % of the first antibacterial protein and 65-85 mole % of the second antibacterial protein.

In another aspect, the antibacterial protein includes 25 mole % of the first antibacterial protein and 75 mole % of the second antibacterial protein.

In another aspect, the concentration of the poloxamer in the mixture before lyophilization is about 0.1 g/L to about 10 g/L.

In another aspect, the poloxamer is poloxamer 188.

In another aspect, the sugar is D-sorbitol.

In another aspect, the concentration of the sugar in the mixture before lyophilization is about 1 g/L to about 600 g/L.

In another aspect, the amino acid is L-histidine.

In another aspect, the concentration of the amino acid in the mixture before lyophilization is about 0.1 g/L to about 10 g/L.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention.

In the drawings:

FIG. 1 is a result of size-exclusion high-performance liquid chromatography analyzed at time zero for a freeze-dried formulation.

FIG. 2 is a result of size-exclusion high-performance liquid chromatography analyzed after 1 month of storage for a freeze-dried formulation.

FIG. 3 is a result of size-exclusion high-performance liquid chromatography analyzed after 6 months of storage for a freeze-dried formulation.

### DETAILED DESCRIPTION OF THE ILLUSTRATED EMBODIMENTS

Reference will now be made in detail to embodiments of the present invention, example of which is illustrated in the accompanying drawings.

As used herein, “at least one of or all the following Staphylococcus species” means any one, two, three, four, five, six . . . up to twenty-two Staphylococcus species selected from the group consisting of Staphylococcus arlettae, Staphylococcus aureus, Staphylococcus auricularis, Staphylococcus carnosus, Staphylococcus carprae, Staphylococcus chromogenes, Staphylococcus cohnii, Staphylococcus delphini, Staphylococcus epidermidis, Staphylococcus equorum, Staphylococcus gallinarum, Staphylococcus hemolyticus, Staphylococcus hominis, Staphylococcus intermedius, Staphylococcus kloosii, Staphylococcus lentus, Staphylococcus lugdunensis, Staphylococcus muscae, Staphylococcus pasteuri, Staphylococcus saprophyticus, Staphylococcus warneri, and Staphylococcus xylosus.

It is known that proteins are relatively unstable in aqueous state and undergo chemical and physical degradation resulting in a loss of biological activity during processing and storage. Freeze-drying (also known as lyophilisation) is a method for preserving proteins for storage.

A freeze-dried formulation includes an antibacterial protein having killing activity specific to at least one of or all following species: Staphylococcus arlettae, Staphylococcus aureus, Staphylococcus auricularis, Staphylococcus carnosus, Staphylococcus carprae, Staphylococcus chromogenes, Staphylococcus cohnii, Staphylococcus delphini, Staphylococcus epidermidis, Staphylococcus equorum, Staphylococcus gallinarum, Staphylococcus hemolyticus, Staphylococcus hominis, Staphylococcus intermedius, Staphylococcus kloosii, Staphylococcus lentus, Staphylococcus lugdunensis, Staphylococcus muscae, Staphylococcus pasteuri, Staphylococcus saprophyticus, Staphylococcus warneri, and Staphylococcus xylosus; a poloxamer; a sugar; and an amino acid.

A method for manufacturing a freeze-dried formulation includes forming a mixture consisting of an antibacterial protein having killing activity specific to at least one of or all following species: Staphylococcus arlettae, Staphylococcus aureus, Staphylococcus auricularis, Staphylococcus carnosus, Staphylococcus carprae, Staphylococcus chromogenes, Staphylococcus cohnii, Staphylococcus delphini, Staphylococcus epidermidis, Staphylococcus equorum, Staphylococcus gallinarum, Staphylococcus hemolyticus, Staphylococcus hominis, Staphylococcus intermedius, Staphylococcus kloosii, Staphylococcus lentus, Staphylococcus lugdunensis, Staphylococcus muscae, Staphylococcus pasteuri, Staphylococcus saprophyticus, Staphylococcus warneri, and Staphylococcus xylosus; a poloxamer; a sugar; and an amino acid, and subjecting the mixture to lyophilization.

The concentration of the antibacterial protein in solution before freeze-drying can be from about 0.1 mg/mL to about 30 mg/mL, from 0.1 mg/mL to 30 mg/mL, from 0.5 mg/mL to 30 mg/mL, from 1.0 mg/mL to 30 mg/mL, from 1.5 mg/mL to 30 mg/mL, from 5 mg/mL to 30 mg/mL, from 0.1 mg/mL to 25 mg/mL, from 0.1 mg/mL to 20 mg/mL, from 0.5 mg/mL to 25 mg/mL, from 0.5 mg/mL to 20 mg/mL, or from 1.0 mg/mL to 20 mg/mL.

The antibacterial protein consists of the amino acid sequence of SEQ. ID. NO: 1, consists of the amino acid sequence of SEQ. ID. NO: 2, or is a mixture of a first antibacterial protein consisting of the amino acid sequence of SEQ. ID. NO: 1 and a second antibacterial protein consisting of the amino acid sequence of SEQ. ID. NO: 2.

When the antibacterial protein is a mixture of the first antibacterial protein and the second antibacterial protein, the antibacterial protein can include 15-35 mole % of the first antibacterial protein and 65-85 mole % of the second antibacterial protein. For example, the antibacterial protein includes 15, 16, 17, 18, 19, 20, 21, 22, 23, 24, 25, 26, 27, 28, 29, 30, 31, 32, 33, 34, or 35 mole % of the first antibacterial protein, and 65, 66, 67, 68, 69, 70, 71, 72, 73, 74, 75, 76, 77, 78, 79, 80, 81, 82, 83, 84, or 85 mole % of the second antibacterial protein.

Poloxamers are nonionic triblock copolymers composed of a central hydrophobic chain of polyoxypropylene (poly(propylene oxide)) and two hydrophilic chains of polyoxyethylene (poly(ethylene oxide)). The concentration of the poloxamer in solution before freeze-drying can be about 0.1 g/L to about 10 g/L, 0.1 g/L to 10 g/L, 0.2 g/L to 10 g/L, 0.1 g/L to 8 g/L, 0.2 g/L to 8 g/L, 0.1 g/L to 6 g/L, or 0.2 g/L to 6 g/L. Preferably, the poloxamer is poloxamer 188.

Preferred sugars used in the freeze-dried formulation are, for example, D-sorbitol, sucrose, glucose, lactose, trehalose, glycerol, ethylene glycol, mannitol, xylitol and inositol. More preferably, the sugar is D-sorbitol. The concentration of the sugar in solution before freeze-drying can be about 1 g/L to about 600 g/L, 1 g/L to 600 g/L, 5 g/L to 600 g/L, 1 g/L to 500 g/L, 5 g/L to 500 g/L, 1 g/L to 400 g/L, or 5 g/L to 400 g/L.

Preferred amino acids used in the freeze-dried formulation are, for example, L-histidine, L-glycine, and L-arginine. More preferably, the amino acid is L-histidine. The concentration of the amino acid in solution before freeze-drying can be about 0.1 g/L to about 10 g/L, 0.1 g/L to 10 g/L, 0.5 g/L to 10 g/L, 0.1 g/L to 8 g/L, 0.5 g/L to 8 g/L, 0.1 g/L to 6 g/L, or 0.5 g/L to 6 g/L.

An antibacterial formulation includes an antibacterial protein having killing activity specific to at least one of or all following species: Staphylococcus arlettae, Staphylococcus aureus, Staphylococcus auricularis, Staphylococcus carnosus, Staphylococcus carprae, Staphylococcus chromogenes, Staphylococcus cohnii, Staphylococcus delphini, Staphylococcus epidermidis, Staphylococcus equorum, Staphylococcus gallinarum, Staphylococcus hemolyticus, Staphylococcus hominis, Staphylococcus intermedius, Staphylococcus kloosii, Staphylococcus lentus, Staphylococcus lugdunensis, Staphylococcus muscae, Staphylococcus pasteuri, Staphylococcus saprophyticus, Staphylococcus warneri, and Staphylococcus xylosus; a poloxamer; a sugar; an amino acid; and water. The antibacterial protein consists of the amino acid sequence of SEQ. ID. NO: 1, consists of the amino acid sequence of SEQ. ID. NO: 2, or includes a first antibacterial protein consisting of the amino acid sequence of SEQ. ID. NO: 1 and a second antibacterial protein consisting of the amino acid sequence of SEQ. ID. NO: 2.

The concentration of the antibacterial protein in the antibacterial formulation can be from about 0.1 mg/mL to about 30 mg/mL, from 0.1 mg/mL to 30 mg/mL, from 0.5 mg/mL to 30 mg/mL, from 1.0 mg/mL to 30 mg/mL, from 1.5 mg/mL to 30 mg/mL, from 5 mg/mL to 30 mg/mL, from 0.1 mg/mL to 25 mg/mL, from 0.1 mg/mL to 20 mg/mL, from 0.5 mg/mL to 25 mg/mL, from 0.5 mg/mL to 20 mg/mL, or from 1.0 mg/mL to 20 mg/mL.

When the antibacterial protein is a mixture of the first antibacterial protein and the second antibacterial protein, the antibacterial protein can include 15-35 mole % of the first antibacterial protein and 65-85 mole % of the second antibacterial protein. For example, the antibacterial protein includes 15, 16, 17, 18, 19, 20, 21, 22, 23, 24, 25, 26, 27, 28, 29, 30, 31, 32, 33, 34, or 35 mole % of the first antibacterial protein, and 65, 66, 67, 68, 69, 70, 71, 72, 73, 74, 75, 76, 77, 78, 79, 80, 81, 82, 83, 84, or 85 mole % of the second antibacterial protein.

The concentration of the poloxamer in the antibacterial formulation can be about 0.1 g/L to about 10 g/L, 0.1 g/L to 10 g/L, 0.2 g/L to 10 g/L, 0.1 g/L to 8 g/L, 0.2 g/L to 8 g/L, 0.1 g/L to 6 g/L, or 0.2 g/L to 6 g/L. Preferably, the poloxamer is poloxamer 188.

Preferred sugars used in the antibacterial formulation are, for example, D-sorbitol, sucrose, glucose, lactose, trehalose, glycerol, ethylene glycol, mannitol, xylitol and inositol. The concentration of the sugar in the antibacterial formulation can be about 1 g/L to about 600 g/L, 1 g/L to 600 g/L, 5 g/L to 600 g/L, 1 g/L to 500 g/L, 5 g/L to 500 g/L, 1 g/L to 400 g/L, or 5 g/L to 400 g/L.

Preferred amino acids used in the antibacterial formulation are, for example, L-histidine, L-glycine, and L-arginine. More preferably, the amino acid is L-histidine. The concentration of the amino acid in the antibacterial formulation can be about 0.1 g/L to about 10 g/L, 0.1 g/L to 10 g/L, 0.5 g/L to 10 g/L, 0.1 g/L to 8 g/L, 0.5 g/L to 8 g/L, 0.1 g/L to 6 g/L, or 0.5 g/L to 6 g/L.

A method for manufacturing a freeze-dried formulation includes forming a mixture consisting of an antibacterial protein having killing activity specific to at least one of or all following species: Staphylococcus arlettae, Staphylococcus aureus, Staphylococcus auricularis, Staphylococcus carnosus, Staphylococcus carprae, Staphylococcus chromogenes, Staphylococcus cohnii, Staphylococcus delphini, Staphylococcus epidermidis, Staphylococcus equorum, Staphylococcus gallinarum, Staphylococcus hemolyticus, Staphylococcus hominis, Staphylococcus intermedius, Staphylococcus kloosii, Staphylococcus lentus, Staphylococcus lugdunensis, Staphylococcus muscae, Staphylococcus pasteuri, Staphylococcus saprophyticus, Staphylococcus warneri, and Staphylococcus xylosus; a poloxamer; a sugar; and an amino acid, and subjecting the mixture to lyophilization.

Practical and presently preferred embodiments of the present invention are illustrative as shown in the following Examples.

However, it will be appreciated that those skilled in the art, on consideration of this disclosure, may make modifications and improvements within the spirit and scope of the present invention.

### Example 1: Preparation of the Antibacterial Protein

An expression plasmid of the antibacterial protein of the present invention was constructed by conventional subcloning a gene encoding the antibacterial protein of the present invention, which is presented by SEQ. ID. NO: 3, into the pBAD-TOPO vector (Invitrogen). Escherichia coli BL21 cell transformed with the resultant plasmid was used as a production host for the antibacterial protein of the present invention.

Expression of the antibacterial protein of the present invention was induced with 0.2% arabinose at an optical density at 600 nm (OD₆₀₀) of 2.0 and the induced bacterial cells were subsequently incubated for an additional 10 hours at 19° C. Bacterial cells were recovered by centrifugation (6,000×g for 20 minutes) and the resulting cell pellet was re-suspended in lysis buffer [50 mM Na₂HPO₄ (pH 7.5), 10 mM ethylene diamine tetra-acetic acid (EDTA), 1 mM dithiothreitol (DTT)] and disrupted using a conventional ultrasonic treatment for 5 minutes (1 second pulse with 3 seconds rest interval between pulses). Following centrifugation (13,000×g for 20 minutes), the supernatant was recovered and subjected to two-step chromatography comprising ion exchange chromatography (SP fast flow column; GE Healthcare) and hydrophobic interaction chromatography (Toyopearl PPG-600M column; Tosoh Bioscience).

To be more descriptive, the prepared production host was inoculated in a TSB (tryptic soy broth) medium (casein digest, 17 g/L; soybean digest, 3 g/L; dextrose, 2.5 g/L; NaCl, 5 g/L; dipotassium phosphate, 2.5 g/L), and incubation at 37° C. was performed. When the cell concentration reached 2.0 of OD₆₀₀, L-arabinose was added at the final concentration of 0.2% to induce the expression of the antibacterial protein. The cells were cultured at 19° C. for 10 more hours from the point of induction. The culture broth was centrifuged at 6,000×g for 20 minutes to obtain cell precipitate. The precipitate was suspended in 50 mM Na₂HPO₄ buffer (pH 7.5) containing 10 mM EDTA and 1 mM DTT (10 mL of buffer per 1 g of cells). Cells in the suspension were disrupted by conventional sonication. The cell lysate was centrifuged at 13,000×g for 20 minutes to remove the cell debris. The supernatant precipitate was subjected to the two-step chromatography comprising ion exchange chromatography (Buffer A: 25 mM Na₂HPO₄ (pH 7.5), 10 mM EDTA; Buffer B: 25 mM Na₂HPO₄ (pH 7.5), 10 mM EDTA, 1 M NaCl; Buffer C: 25 mM Na₂HPO₄ (pH 7.5), 10 mM EDTA, 50 mM NaCl, 0.5% Triton X-100; Procedure: sample loading→1.6 CV of buffer A→30 CV of buffer C→20 CV of buffer A→5 CV of 22% buffer B→elution by gradient (20 CV of 22-100% buffer B)) and hydrophobic interaction chromatography (Buffer A: 10 mM L-histidine (pH 7.5), 1 M NaCl; Buffer B: 10 mM L-histidine (pH 7.5), 1 M urea; Procedure: sample loading (sample purified by ion exchange chromatography)→10 CV of buffer A→elution by gradient (10 CV of 0-100% buffer B)). The protein solution was then filtered with 0.2 μm filter.

To determine the composition of the antibacterial proteins consisting of the amino acid sequence of SEQ. ID. NO: 1 and SEQ. ID. NO: 2, two-step analysis was performed. First, liquid chromatography (LC)-mass spectrometry (MS) was performed using a protease-treated protein sample. The protein solution obtained according to the procedure described above was subjected to buffer exchange via centrifugal filtration into 50 mM Tris-HCl buffer (pH 7.6) and diluted to a concentration of 2.5 mg/mL with 6 M urea solution. The diluted protein solution was subjected to treatment with protease. As protease, sequencing-grade modified porcine Glu-C protease (Promega, Madison, Wis., USA) was used and the protease treatment was performed according to manufacturer's protocol. After protease treatment, the protease-treated protein solution obtained was subjected to reverse-phase HPLC and Q-TOF-MS. Through peak analysis, the HPLC and MS peaks corresponding to peptide fragment of MAKTQAE originated from the antibacterial protein consisting of the amino acid sequence of SEQ. ID. NO: 1 and peptide fragment of AKTQAE originated from the antibacterial protein consisting of the amino acid sequence of SEQ. ID. NO: 2 were identified based on the estimated protease digestion pattern and mass calculations. In addition, the HPLC and MS peaks were confirmed by comparing the peak pattern obtained using chemically synthesized peptides (MAKTQAE and AKTQAE) as samples. Subsequently, the composition ratio of the antibacterial protein consisting of the amino acid sequence of SEQ. ID. NO: 1 in the antibacterial protein preparation was determined by reverse-phase HPLC analysis with the protease-treated protein sample and chemically synthesized peptides (MAKTQAE and AKTQAE) based on correlation of concentration of peptide and peak area corresponding to it. As results of analysis with three batches of antibacterial protein, the composition ratio of the antibacterial protein consisting of the amino acid sequence of SEQ. ID. NO: 1 was determined to be 25, 27 and 29 mole %.

### Example 2: Preparation of the Pharmaceutical Composition with Freeze-Dried Formulation

A pharmaceutical composition for the treatment of staphylococcal infections comprising the antibacterial proteins of the present invention was prepared by freeze-drying. A freeze dried formulation having the following composition has been prepared:

TABLE 1
Formulation

| | | |
|---|---|---|
| Antibacterial protein | 18 | mg/vial |
| Poloxamer 188 | 1 | mg/vial |
| D-sorbitol | 50 | mg/vial |
| L-histidine | 1.55 | mg/vial |
| CaCl₂•2H₂O | 1.47 | mg/vial |

The manufacturing process consists in buffer exchanging the protein solution prepared in Example 1 into buffer containing the ingredients, concentrating the solution obtained, adjusting the concentration of antibacterial protein in the solution, filtrating the concentration-adjusted solution and lyophilizing the filtrated.

A description of each step of the process is given in the following:
Buffer exchanging the protein solution prepared in Example 1 into buffer (1.56 g/L L-histidine (pH 6.0), 50 g/L D-sorbitol, 1.47 g/L CaCl₂.2H₂O, and 1 g/L poloxamer 188) using conventional diafiltration.
Concentrating the solution obtained using a centrifugal filter (10 K).
Adjusting the concentration of antibacterial protein with buffer (1.56 g/L L-histidine (pH 6.0), 50 g/L D-sorbitol, 1.47 g/L CaCl₂.2H₂O, and 1 g/L poloxamer 188) to be 18 mg/mL based on the protein concentration determined by a conventional bicinchoninic acid (BCA) assay.
Filtrating the concentration-adjusted solution using a 0.2-μm filter.
Adding 1 mL of the filtrated solution in a 3-mL glass vial and placing the filled vial into a stainless steel tray.
Loading the tray into the freeze dryer and lyophilizing the product using the following freeze drying cycle:
   Equilibrating at 4° C. for about 20 minutes.
   Bringing the shelf temperature at −40° C. and maintaining for 12 hours.
   Bringing the condenser temperature at −50° C.
   Applying vacuum to the chamber.
   When the vacuum reaches a value of 1,500 mtorr, raising shelf temperature up to −20° C. and maintaining for 16 hours.
   Raising the shelf temperature up to 20° C. in the manner of increase of 10° C. per 1 hour and maintaining for 4 hours.
   Breaking the vacuum.
   Stoppering and sealing the stoppered vials with the appropriate flip-off caps.

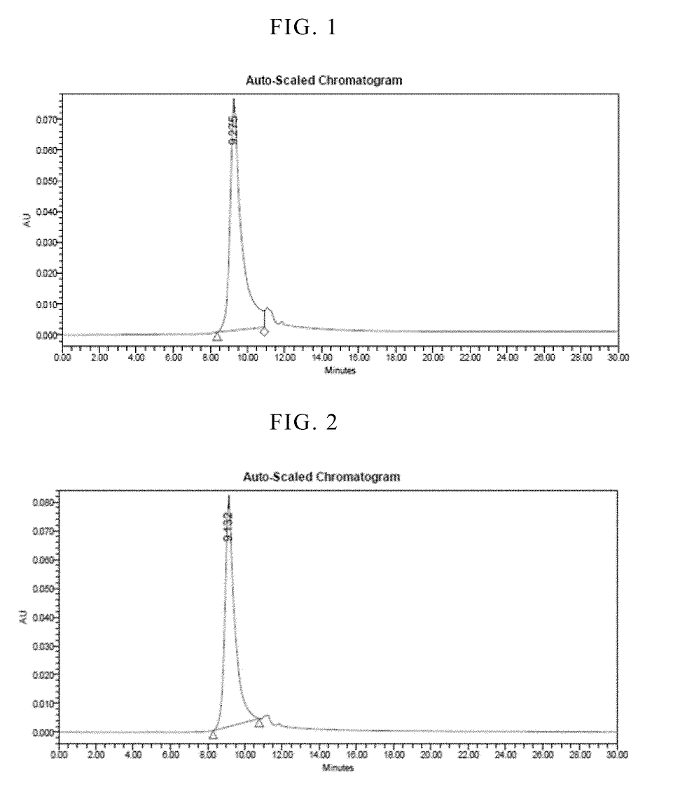

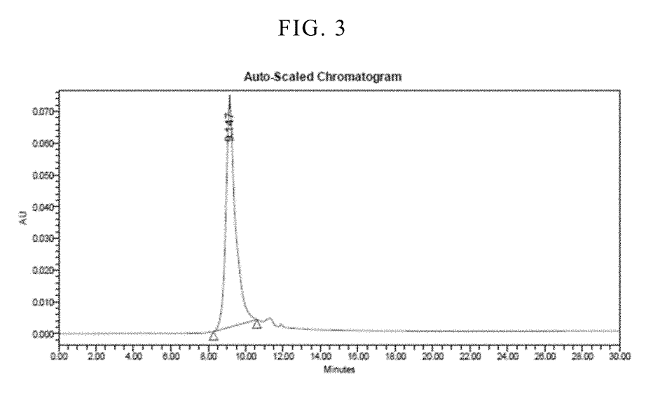

The freeze-dried formulation were stored at 4° C., and tested for stability and biological activity as pointed out below. Prior to analyzing the composition, it was reconstituted using water for injection (0.92 mL). The stability was determined using size-exclusion high-performance liquid chromatography (SEC-HPLC). SEC-HPLC was performed with a BioSep™-SEC-S 2000 column (Phenomenex, Torrance, Calif.). The mobile phase (10 mM Tris, 0.5 M NaCl, 1 M urea, pH 7.5) was applied at a flow rate of 1.0 mL/min. 50 μL sample was injected and sample elutions were monitored for 30 min by measuring absorbance at 280 nm. The results are shown in FIGS. 1-3.

The biological activity was assayed using turbidity reduction assay. The turbidity reduction assay was performed as the follows: the sample was added to suspension of Staphylococcus aureus strain ATCC 33591 (OD₆₀₀=0.5) in 10 mM phosphate-buffered saline (PBS) (pH 7.2) to be a final antibacterial protein concentration of 0.1 μm/mL. Changes in bacterial cell density (OD₆₀₀) were recorded every 30 seconds for 15 minutes. From this experiment, TOD₅₀ (a one-half log drop in the initial concentration of viable bacteria in minutes) was obtained.

Table 2 summarizes the results of the analytical tests related to stability and biological activity of formulation. The values were determined at 4 check-points: at time zero, after 1 month, 3 months and 6 months of storage, at a storage temperature of 4° C. In stability test, the intact protein amount at time zero was considered as 100%. In biological activity test, the difference from the TOD₅₀ value determined at time zero was analyzed.

TABLE 2
Stability and biological activity

| Test | Time zero | 1 Month | 3 Months | 6 Months |
|---|---|---|---|---|
| % Intact protein | 100 | 99.8 | 99.9 | 99.8 |
| amount | | | | |
| % Difference in | 0 | <5 | <5 | <5 |
| biological activity | | | | |

From Table 2 it may be concluded that the stability and biological activity of the freeze-dried formulation of the present invention are well conserved after 6 months of storage.

### Example 3: Comparison of the Freeze-Dried Formulation and Liquid Formulation

Biological activity of the freeze-dried formulation and liquid formulation was compared using the turbidity reduction assay used in Example 2. As freeze-dried formulation, 1-month stored freeze-dried formulation was used. Prior to analyzing the biological activity, it was reconstituted using water for injection (0.92 mL). As liquid formulation, the filtrated solution freshly prepared according to the procedure described in Example 2 was used. In this experiment, the following strains were used.

TABLE 3
Test Strains

| | | | Antibiotic |
|---|---|---|---|
| | | Strain | resistance |
| No. | Species | information | information |
| 1 | Staphylococcus arlettae | KCTC 3588 | Not available |
| | | (ATCC 43957) | |
| 2 | Staphylococcus aureus | ATCC 35556 | Not available |
| 3 | Staphylococcus auricularis | KCTC 3584 | Not available |
| | | (ATTC 33753) | |
| 4 | Staphylococcus carnosus | KCTC 3580 | Not available |
| | | (ATCC 51365) | |
| 5 | Staphylococcus carprae | KCTC 3583 | Not available |
| | | (ATCC 35538) | |
| 6 | Staphylococcus chromogenes | KCTC 3579 | Not available |
| | | (ATCC 43764) | |
| 7 | Staphylococcus cohnii | KCTC 3574 | Not available |
| | | (ATCC 49330) | |
| 8 | Staphylococcus delphini | KCTC 3592 | Not available |
| | | (ATCC 49171) | |
| 9 | Staphylococcus epidermidis | CCARM 3751 | Ampicillin |
| | | | resistant; |
| | | | Clindamycin |
| | | | resistant; |
| | | | Erythromycin |
| | | | resistant; |
| | | | Gentamycin |
| | | | resistant |
| 10 | Staphylococcus equorum | KCTC 3589 | Not available |
| | | (ATCC 43958) | |
| 11 | Staphylococcus gallinarum | KCTC 3585 | Not available |
| | | (ATCC 35539) | |
| 12 | Staphylococcus hemolyticus | CCARM 3733 | Not available |
| 13 | Staphylococcus hominis | CCARM 3732 | Ciprofloxacin |
| | | | resistant |
| 14 | Staphylococcus intermedius | KCTC 3344 | Not available |
| | | (ATCC 29663) | |
| 15 | Staphylococcus kloosii | KCTC 3590 | Not available |
| | | (ATCC 43959) | |
| 16 | Staphylococcus lentus | KCTC 3577 | Not available |
| | | (ATCC 29070) | |
| 17 | Staphylococcus lugdunensis | CCARM 3734 | Not available |
| 18 | Staphylococcus muscae | KCTC 3576 | Not available |
| | | (ATCC 49910) | |
| 19 | Staphylococcus pasteuri | KCTC 13167 | Not available |
| 20 | Staphylococcus saprophyticus | CCARM 3736 | Not available |
| 21 | Staphylococcus warneri | KCTC 3340 | Not available |
| | | (ATCC 27836) | |
| 22 | Staphylococcus xylosus | KCTC 3342 | Not available |
| | | (ATCC 29971) | |

ATCC: American Type Culture Collection;
CCARM: Culture Collection of Antimicrobial Resistant Microbes;
KCTC: Korean Collection for Type Culture

In turbidity reduction assay, the applied final antibacterial protein concentration was 0.1 μg/mL for the following strains: Staphylococcus aureus, Staphylococcus auricularis, Staphylococcus carnosus, Staphylococcus carprae, Staphylococcus chromogenes, Staphylococcus delphini, Staphylococcus epidermidis, Staphylococcus equorum, Staphylococcus gallinarum, Staphylococcus hemolyticus, Staphylococcus hominis, Staphylococcus kloosii, Staphylococcus lugdunensis, Staphylococcus muscae, Staphylococcus saprophyticus, and Staphylococcus xylosus. For the testing against Staphylococcus arlettae, Staphylococcus cohnii, Staphylococcus intermedius, Staphylococcus lentus and Staphylococcus warneri, the applied final antibacterial protein concentration was 0.5 μg/mL. For the testing against Staphylococcus pasteuri, the applied final antibacterial protein concentration was 1.0 μg/mL. The TOD₅₀ value difference was compared between two formulations. The result is provided in Table 4.

TABLE 4

| | Freeze-dried | Liquid |
|---|---|---|
| Species | formulation | formulation |
| Staphylococcus arlettae | 13.0 | 13.1 |
| Staphylococcus aureus | 4.2 | 4.3 |
| Staphylococcus auricularis | 9.1 | 9.1 |
| Staphylococcus carnosus | 20.1 | 20.2 |
| Staphylococcus carprae | 13.2 | 13.2 |
| Staphylococcus chromogenes | 10.5 | 10.6 |
| Staphylococcus cohnii | 15.1 | 15.0 |
| Staphylococcus delphini | 4.6 | 4.6 |
| Staphylococcus epidermidis | 7.3 | 7.5 |
| Staphylococcus equorum | 19.6 | 19.5 |
| Staphylococcus gallinarum | 18.0 | 18.0 |
| Staphylococcus hemolyticus | 8.3 | 8.4 |
| Staphylococcus hominis | 13.4 | 13.3 |
| Staphylococcus intermedius | 9.1 | 9.2 |
| Staphylococcus kloosii | 19.6 | 19.6 |
| Staphylococcus lentus | 10.7 | 10.8 |
| Staphylococcus lugdunensis | 7.5 | 7.5 |
| Staphylococcus muscae | 9.4 | 9.3 |
| Staphylococcus pasteuri | 11.6 | 11.6 |
| Staphylococcus saprophyticus | 5.5 | 5.4 |
| Staphylococcus warneri | 6.2 | 6.3 |
| Staphylococcus xylosus | 13.0 | 13.0 |

The result shown in Table 4 obviously indicates that the freeze-dried formulation of the present invention can provide the very similar antibacterial activity and effectiveness in antibacterial property to liquid formulation. In addition, the result shown in Table 4 shows that the freeze-dried formulation of the present invention has rapid and effective bactericidal activity against various Staphylococcus strains. TOD₅₀ of the freeze-dried formulation of the present invention was less than 20 minutes against almost Staphylococcus strains tested.

In the meantime, the antibacterial activity of the freeze-dried formulation of the present invention against non-Staphylococcus strains was examined. As non-Staphylococcus strains, 2 Enterococcus faecalis strains, 3 Enterococcus faecium strains, 2 Streptococcus mitis strains, 1 Streptococcus uberis strain, 5 Escherichia coli strains, 2 Clostridium perfringens strains and 3 Salmonella strains were tested. As a result, the freeze-dried formulation of the present invention did not have the antibacterial activity against these non-Staphylococcus strains tested (Table 5). This result indicates that the antibacterial activity of the freeze-dried formulation of the present invention is specific to Staphylococcus.

TABLE 5
Antibacterial activity against non-Staphylococcus strains

| | | Test result of | |
|---|---|---|---|
| | | antibacterial activity | |
| | | | Turbidity |
| | | Spot-on-lawn | reduction |
| Bacteria tested | | assay | assay |
| Enterococcus faecalis | Strain 1 | — | — |
| | Strain 2 | — | — |
| Enterococcus faecium | Strain 1 | — | — |
| | Strain 2 | — | — |
| | Strain 3 | — | — |
| Streptococcus mitis | Strain 1 | — | — |
| | Strain 2 | — | — |
| Streptococcus uberis | Strain 1 | — | — |
| Escherichia coli | Strain 1 | — | — |
| | Strain 2 | — | — |
| | Strain 3 | — | — |
| | Strain 4 | — | — |
| | Strain 5 | — | — |
| Clostridium perfringens | Strain 1 | — | — |
| | Strain 2 | — | — |
| Salmonella | Strain 1 | — | — |
| | Strain 2 | — | — |
| | Strain 3 | — | — |

—, No activity.

Therefore, it is concluded that the freeze-dried formulation of the present invention was Staphylococcus specific and has a broad antibacterial spectrum within Staphylococcus, suggesting that the freeze-dried formulation of the present invention can be used as a therapeutic agent for staphylococcal infections.

### Example 4: Therapeutic Effect of the Freeze-Dried Formulation on Single Staphylococcal Infection

Therapeutic effect of the freeze-dried formulation of the present invention on single staphylococcal infections was investigated using animal model. In this experiment, Staphylococcus epidermidis and Staphylococcus hemolyticus were selected as model Staphylococcus strains. As freeze-dried formulation, 1-month stored freeze-dried formulation was used. Prior to use in animal experiment, it was reconstituted using water for injection (0.92 mL). As liquid formulation, the filtrated solution freshly prepared according to the procedure described in Example 2 was used.

For Staphylococcus epidermidis experiment, female ICR mice [specific pathogen-free (SPF) grade] weighing 23 g±20% (5 weeks of age) were used. In total, 30 mice divided into three groups (10 mice per group) were injected intravenously with inocula of Staphylococcus epidermidis strain CCARM 3751 (1×10⁸ CFU/mouse). To the animal of one group (i.e., control group), only buffer (1.56 g/L L-histidine (pH 6.0), 50 g/L D-sorbitol, 1.47 g/L CaCl₂.2H₂O, and 1 g/L poloxamer 188) was administered intravenously three times at 30 minutes, 12 hours, and 24 hours after the bacterial challenge. To the animal of treatment group with the reconstituted solution of freeze-dried formulation, the reconstituted solution of freeze-dried formulation was administered intravenously (dose: 25 mg/kg) three times at 30 minutes, 12 hours, and 24 hours after the bacterial challenge. To the animal of treatment group with the liquid formulation, the liquid formulation was administered intravenously (dose: 25 mg/kg) three times at 30 minutes, 12 hours, and 24 hours after the bacterial challenge. The number of dead mice was recorded and clinical signs were observed daily. The ability of the reconstituted solution of freeze-dried formulation and liquid formulation to eradicate bacteria from the bloodstream was examined using blood collected 5 days after the bacterial challenge (experimental endpoint) by conventional colony counting.

For Staphylococcus hemolyticus experiment, female ICR mice [specific pathogen-free (SPF) grade] weighing 22 g±20% (5 weeks of age) were used. In total, 30 mice divided into three groups (10 mice per group) were injected intravenously with inocula of Staphylococcus hemolyticus strain CCARM 3733 (1×10⁸ CFU/mouse). To the animal of one group (i.e., control group), only buffer (1.56 g/L L-histidine (pH 6.0), 50 g/L D-sorbitol, 1.47 g/L CaCl₂.2H₂O, and 1 g/L poloxamer 188) was administered intravenously three times at 30 minutes, 12 hours, and 24 hours after the bacterial challenge. To the animal of treatment group with the reconstituted solution of freeze-dried formulation, the reconstituted solution of freeze-dried formulation was administered intravenously (dose: 25 mg/kg) three times at 30 minutes, 12 hours, and 24 hours after the bacterial challenge. To the animal of treatment group with the liquid formulation, the liquid formulation was administered intravenously (dose: 25 mg/kg) three times at 30 minutes, 12 hours, and 24 hours after the bacterial challenge. The number of dead mice was recorded and clinical signs were observed daily. The ability of the reconstituted solution of freeze-dried formulation and liquid formulation to eradicate bacteria from the bloodstream was examined using blood collected 5 days after the bacterial challenge (experimental endpoint) by conventional colony counting.

As results, obvious therapeutic effects were observed. Two experiments showed similar results. Regarding clinical signs, although mice in treatment groups were normal for the entire experimental period, mice in control groups showed various clinical signs beginning 2 days after the bacterial challenge, including erythema of the lid margin, decreased locomotor activity, loss of fur, piloerection and circling. Intravenous injections of the reconstituted solution of freeze-dried formulation and liquid formulation significantly increased the survival rate (Table 6).

TABLE 6
Mortality in single staphylococcal infection model experiments

| | | Number of deaths | | | | | | |
|---|---|---|---|---|---|---|---|---|
| | | Days after | | | | | | |
| | | bacterial challenge | | | | | No. dead/ | Mortality |
| Experiment | Group | 1 | 2 | 3 | 4 | 5 | No. challenged | (%) |
| S. epidermidis | Control | 0 | 2 | 3 | 1 | 0 | 6/10 | 60 |
| | Treatment with the | 0 | 0 | 0 | 0 | 0 | 0/10 | 0 |
| | reconstituted | | | | | | | |
| | solution of freeze- | | | | | | | |
| | dried formulation | | | | | | | |
| | Treatment with | 0 | 0 | 0 | 0 | 0 | 0/10 | 0 |
| | liquid formulation | | | | | | | |
| S. hemolyticus | Control | 0 | 2 | 2 | 1 | 0 | 5/10 | 50 |
| | Treatment with the | 0 | 0 | 0 | 0 | 0 | 0/10 | 0 |
| | reconstituted | | | | | | | |
| | solution of freeze- | | | | | | | |
| | dried formulation | | | | | | | |
| | Treatment with | 0 | 0 | 0 | 0 | 0 | 0/10 | 0 |
| | liquid formulation | | | | | | | |

In addition, intravenous injections of the reconstituted solution of freeze-dried formulation and liquid formulation significantly reduced the bacterial counts in blood. The mean CFU/mL was >1×10⁶ in serum collected from the mice of the control group in the Staphylococcus epidermidis experiment and >1×10⁵ in serum from the mice of the control group in the Staphylococcus hemolyticus experiment, whereas no bacterial colonies were observed in mice of all treatment groups.

From the above results, it was confirmed that the freeze-dried formulation of the present invention can provide the very similar therapeutic effect in treating single staphylococcal infections to liquid formulation. In addition, the result shown in Table 6 shows that the freeze-dried formulation of the present invention can be efficiently used for the treatment of staphylococcal infections.

### Example 5: Therapeutic Effect of the Freeze-Dried Formulation on Multiple Staphylococcal Infection

Therapeutic effect of the freeze-dried formulation of the present invention on multiple staphylococcal infections was investigated using animal model. In this experiment, Staphylococcus epidermidis, Staphylococcus lugdunensis and Staphylococcus warneri were selected as model Staphylococcus strains. As freeze-dried formulation, 1-month stored freeze-dried formulation was used. Prior to use in animal experiment, it was reconstituted using water for injection (0.92 mL). As liquid formulation, the filtrated solution freshly prepared according to the procedure described in Example 2 was used.

Female ICR mice [specific pathogen-free (SPF) grade] weighing 22 g±20% (5 weeks of age) were used. In total, 30 mice divided into three groups (10 mice per group) were injected intravenously with mixed inocula of Staphylococcus epidermidis CCARM 3751, Staphylococcus lugdunensis CCARM 3734 and Staphylococcus warneri KCTC 3340 (ATCC 27836) (1×10⁸ CFU each/mouse). To the animal of one group (i.e., control group), only buffer (1.56 g/L L-histidine (pH 6.0), 50 g/L D-sorbitol, 1.47 g/L CaCl₂.2H₂O, and 1 g/L poloxamer 188) was administered intravenously three times at 30 minutes, 12 hours, and 24 hours after the bacterial challenge. To the animal of treatment group with the reconstituted solution of freeze-dried formulation, the reconstituted solution of freeze-dried formulation was administered intravenously (dose: 25 mg/kg) three times at 30 minutes, 12 hours, and 24 hours after the bacterial challenge. To the animal of treatment group with the liquid formulation, the liquid formulation was administered intravenously (dose: 25 mg/kg) three times at 30 minutes, 12 hours, and 24 hours after the bacterial challenge. The number of dead mice was recorded and clinical signs were observed daily. The ability of the reconstituted solution of freeze-dried formulation and liquid formulation to eradicate bacteria from the bloodstream was examined using blood collected 5 days after the bacterial challenge (experimental endpoint) by conventional colony counting.

As results, obvious therapeutic effects were observed. Regarding clinical signs, although mice in treatment groups were normal for the entire experimental period, mice in control group showed various clinical signs, including erythema of the lid margin, decreased locomotor activity, loss of fur, ptosis, and piloerection. Intravenous injections of the reconstituted solution of freeze-dried formulation and liquid formulation significantly increased the survival rate (Table 7).

TABLE 7
Mortality in multiple staphylococcal infection model experiment

| | Number of deaths | | | | | | |
|---|---|---|---|---|---|---|---|
| | Days after | | | | | | |
| | bacterial challenge | | | | | No. dead/ | Mortality |
| Group | 1 | 2 | 3 | 4 | 5 | No. challenged | (%) |
| Control | 0 | 3 | 2 | 2 | 0 | 7/10 | 70 |
| Treatment with the | 0 | 0 | 0 | 0 | 0 | 0/10 | 0 |
| reconstituted | | | | | | | |
| solution of freeze- | | | | | | | |
| dried formulation | | | | | | | |
| Treatment with | 0 | 0 | 0 | 0 | 0 | 0/10 | 0 |
| liquid formulation | | | | | | | |

In addition, intravenous injections of the reconstituted solution of freeze-dried formulation and liquid formulation significantly reduced the bacterial counts in blood. The mean CFU/mL was >1×10⁶ in serum collected from the mice of the control group, whereas no bacterial colonies were observed in mice of all treatment groups.

From the above results, it was confirmed that the freeze-dried formulation of the present invention can provide the very similar therapeutic effect in treating multiple staphylococcal infections to liquid formulation. In addition, the result shown in Table 7 shows that the freeze-dried formulation of the present invention can be efficiently used for the treatment of staphylococcal infections.

It will be apparent to those skilled in the art that various modifications and variations can be made in the present invention without departing from the spirit or scope of the invention. Thus, it is intended that the present invention cover the modifications and variations of this invention provided they come within the scope of the appended claims and their equivalents.

1. A freeze-dried formulation comprising:
an antibacterial protein having killing activity specific to at least one of or all following species: Staphylococcus arlettae, Staphylococcus aureus, Staphylococcus auricularis, Staphylococcus carnosus, Staphylococcus carprae, Staphylococcus chromogenes, Staphylococcus cohnii, Staphylococcus delphini, Staphylococcus epidermidis, Staphylococcus equorum, Staphylococcus gallinarum, Staphylococcus hemolyticus, Staphylococcus hominis, Staphylococcus intermedius, Staphylococcus kloosii, Staphylococcus lentus, Staphylococcus lugdunensis, Staphylococcus muscae, Staphylococcus pasteuri, Staphylococcus saprophyticus, Staphylococcus warneri, and Staphylococcus xylosus,
a poloxamer,
a sugar, and
an amino acid.

2. The freeze-dried formulation of claim 1, wherein the concentration of the antibacterial protein in solution before freeze-drying is about 0.1 mg/mL to about 30 mg/mL.

3. The freeze-dried formulation of claim 1, wherein the antibacterial protein consists of the amino acid sequence of SEQ. ID. NO: 1.

4. The freeze-dried formulation of claim 1, wherein the antibacterial protein consists of the amino acid sequence of SEQ. ID. NO: 2.

5. The freeze-dried formulation of claim 1, wherein the antibacterial protein is a mixture of a first antibacterial protein consisting of the amino acid sequence of SEQ. ID. NO: 1 and a second antibacterial protein consisting of the amino acid sequence of SEQ. ID. NO: 2.

6. The freeze-dried formulation of claim 5, wherein the antibacterial protein includes 15-35 mole % of the first antibacterial protein and 65-85 mole % of the second antibacterial protein.

7. The freeze-dried formulation of claim 6, wherein the antibacterial protein includes 25 mole % of the first antibacterial protein and 75 mole % of the second antibacterial protein.

8. The freeze-dried formulation of claim 1, wherein the concentration of the poloxamer in solution before freeze-drying is about 0.1 g/L to about 10 g/L.

9. The freeze-dried formulation of claim 1, wherein the poloxamer is poloxamer 188.

10. The freeze-dried formulation of claim 1, wherein the sugar is D-sorbitol.

11. The freeze-dried formulation of claim 1, wherein the concentration of the sugar in solution before freeze-drying is about 1 g/L to about 600 g/L.

12. The freeze-dried formulation of claim 1, wherein the amino acid is L-histidine.

13. The freeze-dried formulation of claim 1, wherein the concentration of the amino acid in solution before freeze-drying is about 0.1 g/L to about 10 g/L.

14. An antibacterial formulation comprising:
an antibacterial protein having killing activity specific to at least one of or all following species: Staphylococcus arlettae, Staphylococcus aureus, Staphylococcus auricularis, Staphylococcus carnosus, Staphylococcus carprae, Staphylococcus chromogenes, Staphylococcus cohnii, Staphylococcus delphini, Staphylococcus epidermidis, Staphylococcus equorum, Staphylococcus gallinarum, Staphylococcus hemolyticus, Staphylococcus hominis, Staphylococcus intermedius, Staphylococcus kloosii, Staphylococcus lentus, Staphylococcus lugdunensis, Staphylococcus muscae, Staphylococcus pasteuri, Staphylococcus saprophyticus, Staphylococcus warneri, and Staphylococcus xylosus,
a poloxamer,
a sugar,
an amino acid, and
water
wherein the antibacterial protein consisting of the amino acid sequence of SEQ. ID. NO: 1, and
wherein the concentration of the antibacterial protein is about 0.1 mg/mL to about 30 mg/mL.

15. An antibacterial formulation comprising:
an antibacterial protein having killing activity specific to at least one of or all following species: Staphylococcus arlettae, Staphylococcus aureus, Staphylococcus auricularis, Staphylococcus carnosus, Staphylococcus carprae, Staphylococcus chromogenes, Staphylococcus cohnii, Staphylococcus delphini, Staphylococcus epidermidis, Staphylococcus equorum, Staphylococcus gallinarum, Staphylococcus hemolyticus, Staphylococcus hominis, Staphylococcus intermedius, Staphylococcus kloosii, Staphylococcus lentus, Staphylococcus lugdunensis, Staphylococcus muscae, Staphylococcus pasteuri, Staphylococcus saprophyticus, Staphylococcus warneri, and Staphylococcus xylosus,
a poloxamer,
a sugar,
an amino acid, and
water
wherein the antibacterial protein consisting of the amino acid sequence of SEQ. ID. NO: 2, and
wherein the concentration of the antibacterial protein is about 0.1 mg/mL to about 30 mg/mL.

16. An antibacterial formulation comprising:
an antibacterial protein having killing activity specific to at least one of or all following species: Staphylococcus arlettae, Staphylococcus aureus, Staphylococcus auricularis, Staphylococcus carnosus, Staphylococcus carprae, Staphylococcus chromogenes, Staphylococcus cohnii, Staphylococcus delphini, Staphylococcus epidermidis, Staphylococcus equorum, Staphylococcus gallinarum, Staphylococcus hemolyticus, Staphylococcus hominis, Staphylococcus intermedius, Staphylococcus kloosii, Staphylococcus lentus, Staphylococcus lugdunensis, Staphylococcus muscae, Staphylococcus pasteuri, Staphylococcus saprophyticus, Staphylococcus warneri, and Staphylococcus xylosus,
a poloxamer,
a sugar,
an amino acid, and
water
wherein the antibacterial protein includes a first antibacterial protein consisting of the amino acid sequence of SEQ. ID. NO: 1 and a second antibacterial protein consisting of the amino acid sequence of SEQ. ID. NO: 2, and
wherein the concentration of the antibacterial protein is about 0.1 mg/mL to about 30 mg/mL.

17. The antibacterial formulation of claim 16, wherein the antibacterial protein includes 15-35 mole % of the first antibacterial protein and 65-85 mole % of the second antibacterial protein.

18. The antibacterial formulation of claim 17, wherein the antibacterial protein includes 25 mole % of the first antibacterial protein and 75 mole % of the second antibacterial protein.

19. (canceled)

20. (canceled)

21. (canceled)

22. (canceled)

23. (canceled)

24. (canceled)

25. A method for manufacturing a freeze-dried formulation comprising:
forming a mixture consisting of an antibacterial protein having killing activity specific to at least one of or all following species: Staphylococcus arlettae, Staphylococcus aureus, Staphylococcus auricularis, Staphylococcus carnosus, Staphylococcus carprae, Staphylococcus chromogenes, Staphylococcus cohnii, Staphylococcus delphini, Staphylococcus epidermidis, Staphylococcus equorum, Staphylococcus gallinarum, Staphylococcus hemolyticus, Staphylococcus hominis, Staphylococcus intermedius, Staphylococcus kloosii, Staphylococcus lentus, Staphylococcus lugdunensis, Staphylococcus muscae, Staphylococcus pasteuri, Staphylococcus saprophyticus, Staphylococcus warneri, and Staphylococcus xylosus; a poloxamer; a sugar; and an amino acid; and
subjecting the mixture to lyophilization.

26. The method according to claim 25, wherein the concentration of the antibacterial protein in the mixture before lyophilization is about 0.1 mg/mL to about 30 mg/mL.

27. The method according to claim 25, wherein the antibacterial protein consists of the amino acid sequence of SEQ. ID. NO: 1.

28. The method according to claim 25, wherein the antibacterial protein consists of the amino acid sequence of SEQ. ID. NO: 2.

29. The method according to claim 25, wherein the antibacterial protein is a mixture of a first antibacterial protein consisting of the amino acid sequence of SEQ. ID. NO: 1 and a second antibacterial protein consisting of the amino acid sequence of SEQ. ID. NO: 2.

30. The method according to claim 29, wherein the antibacterial protein includes 15-35 mole % of the first antibacterial protein and 65-85 mole % of the second antibacterial protein.

31. The method according to claim 30, wherein the antibacterial protein includes 25 mole % of the first antibacterial protein and 75 mole % of the second antibacterial protein.

32. (canceled)

33. (canceled)

34. (canceled)

35. (canceled)

36. (canceled)

37. (canceled)


SEQUENCE LISTING (3 sequences)

SEQ ID NO: 1 - protein, 495 aa, Artificial Sequence
  antibacterial composition
MAKTQAEINKRLDAYAKGTVDSPYRIKKATSYDPSFGVMEAGAIDADGYYHAQCQDLITDYVLWLTDNKVRTWGNAKDQIKQSYGTGFKIHENKPSTVPKKGWIAVFTSGSYQQWGHIGIVYDGGNTSTFTILEQNWNGYANKKPTKRVDNYYGLTHFIEIPVKAGTTVKKETAKKSASKTPAPKKKATLKVSKNHINYTMDKRGKKPEGMVIHNDAGRSSGQQYENSLANAGYARYANGIAHYYGSEGYVWEAIDAKNQIAWHTGDGTGANSGNFRFAGIEVCQSMSASDAQFLKNEQAVFQFTAEKFKEWGLTPNRKTVRLHMEFVPTACPHRSMVLHTGFNPVTQGRPSQAIMNKLKDYFIKQIKNYMDKGTSSSTVVKDGKTSSASTPATRPVTGSWKKNQYGTWYKPENATFVNGNQPIVTRIGSPFLNAPVGGNLPAGATIVYDEVCIQAGHIWIGYNAYNGNRVYCPVRTCQGVPPNHIPGVAWGVFK

SEQ ID NO: 2 - protein, 494 aa, Artificial Sequence
  antibacterial composition
AKTQAEINKRLDAYAKGTVDSPYRIKKATSYDPSFGVMEAGAIDADGYYHAQCQDLITDYVLWLTDNKVRTWGNAKDQIKQSYGTGFKIHENKPSTVPKKGWIAVFTSGSYQQWGHIGIVYDGGNTSTFTILEQNWNGYANKKPTKRVDNYYGLTHFIEIPVKAGTTVKKETAKKSASKTPAPKKKATLKVSKNHINYTMDKRGKKPEGMVIHNDAGRSSGQQYENSLANAGYARYANGIAHYYGSEGYVWEAIDAKNQIAWHTGDGTGANSGNFRFAGIEVCQSMSASDAQFLKNEQAVFQFTAEKFKEWGLTPNRKTVRLHMEFVPTACPHRSMVLHTGFNPVTQGRPSQAIMNKLKDYFIKQIKNYMDKGTSSSTVVKDGKTSSASTPATRPVTGSWKKNQYGTWYKPENATFVNGNQPIVTRIGSPFLNAPVGGNLPAGATIVYDEVCIQAGHIWIGYNAYNGNRVYCPVRTCQGVPPNHIPGVAWGVFK

SEQ ID NO: 3 - DNA, 1488 bp, Artificial Sequence
  antibacterial composition
atggctaagactcaagcagaaataaataaacgtttagacgcttatgcaaaaggtacagtagacagtccttatagaattaaaaaagctacaagctatgacccatcgtttggtgtaatggaagcaggagcaattgacgcagatggttactatcatgcacagtgccaagacttaattactgattatgtattatggttaacagataataaagttagaacttggggtaatgctaaagaccaaatcaaacaaagttatggtactggatttaaaatacatgaaaataaaccttctacagtacctaaaaaaggatggattgctgtatttacatccggtagttatcagcaatggggtcacataggtattgtatatgatggaggtaatacttctacatttactattttagagcaaaactggaacggttacgctaataaaaaacctacaaaacgtgtagataattattacggattaactcattttattgagatacctgtaaaagcaggaactactgttaaaaaagaaacagctaagaaaagtgcaagtaaaacacctgcacctaaaaagaaagcaacactaaaagtttctaagaaccatattaactatacaatggataaacgtggtaagaaacctgaaggaatggtaatacacaacgatgcaggtcgttcttcagggcaacaatacgagaattcattagctaacgcaggttatgctagatatgctaatggtattgctcattactatggctctgaaggttatgtatgggaagcaatagatgctaagaatcaaattgcttggcacacaggagatggaacaggagcaaactcaggtaactttagatttgcaggtattgaagtctgtcaatcaatgagtgctagtgatgctcaattccttaaaaacgaacaagcagtattccaatttactgcagagaaatttaaagaatggggtcttactcctaatcgtaaaactgtaagattgcatatggaatttgttccaacagcttgtcctcatcgttctatggttcttcatacaggatttaatccagtaacacaaggaagaccatctcaagcaataatgaataaactaaaagattatttcattaaacaaattaaaaactacatggataaaggaacttcaagttctacagtagttaaagacggtaaaacaagtagcgcaagtacaccggcaactagaccagtaacaggctcttggaaaaagaaccagtacggaacttggtacaaaccggaaaatgcaacatttgttaatggtaaccaacctatagtaactagaataggttctccattcttaaatgctccagtaggaggtaacttaccggcaggagctacaattgtatatgacgaagtttgtatccaagcaggtcacatttggataggttacaatgcttacaatggtaacagagtatattgccctgttagaacttgtcaaggagttccacctaatcatatacctggggttgcctggggagtattcaaatag